(19) United States
(12) Patent Application Publication
(10) Pub. No.: US 2005/0049647 A1
(43) Pub. Date: Mar. 3, 2005

(54) Subcutaneous switch for implantable medical device

(76) Inventor: Walter H. Olson, North Oaks, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/652,839

(22) Filed: Aug. 29, 2003

(51) Int. Cl. A61N 1/375

(52) U.S. Cl. 607/36

(57) ABSTRACT

An externally actuable, hermetically sealed switch is incorporated with an implantable medical device (IMD). A patient applies pressure against the tissue over the IMD and actuates the switch. The actuation of the switch causes the IMD to take predetermined actions, such as recording data, inhibiting therapy, initiating therapy, increasing therapy, requesting information, initiating a communications session, or performing a status check. Thus, the patient is able to interact with the IMD without requiring an external device such as a programmer, patient activator or magnet.

### FIELD OF THE INVENTION

The present invention relates to implantable medical devices. More specifically, the present invention relates to implantable medical devices having features that are patient activated.

DESCRIPTION OF THE RELATED ART

Various implantable medical devices (IMDs) are commonly used to deliver therapies or to monitor physiological parameters. For example, pacemakers are commonly used to manage cardiac rhythms, defibrillators are used restore sinus rhythm to a heart in fibrillation, and implantable monitors, such as the Medtronic Reveal, are used to record data over time.

Some IMDs include features that are actuated by the patient. For example, some cardiac monitors will constantly record data in predetermined, looping increments (e.g., 15 minutes), but will only commit that data to permanent memory if the patient indicates that a notable event has occurred (e.g., syncope). As another example, a patient having atrial fibrillation may choose to delay what may be an uncomfortable defibrillation therapy in hopes that the heart will autonomously restore sinus rhythm. That is, the implanted defibrillator may have a preprogrammed time delay before delivering the therapy, triggered by the detection of atrial fibrillation; however, the patient may signal the device to extend the delay. A patient or caregiver may also query the IMD to determine if it is functioning properly, if the IMD has delivered a suspected therapy, or to determine various other types of information.

In any event, the patient provides an input to the various medical devices to initiate a given action. Typically, such patient communication includes placing a programming head over the IMD and utilizing a programmer to telemeter data to and from the device. Alternatively, the patient may have an RF device that transmits a signal that is received at the IMD to initiate the action. Such a communication device may take various forms, but requires the patient to utilize an external component to communicate with the device. As such, if the patient does not have the external component, communication with the IMD is precluded. Therefore, the patient may not be able to choose therapy options, signal the IMD to record data, request status or operability information from the IMD, or initiate other functionality.

### SUMMARY OF THE INVENTION

The present invention, in one embodiment, is an implantable medical device having a hermetically sealed switch disposed on an exterior portion of the housing. Thus, once implanted, the patient or a caregiver can actuate the switch by pressing against the tissue over the implant site.

There are many actions that a patient may desire to actuate on the implantable medical device. Several have traditionally required an external device, such as a programmer, magnet, RF communications device, etc. With the present invention, the patient will always have the ability to toggle the desired function simply by actuating the switch. The switch can be used to initiate a therapy, inhibit a therapy, initiate a self diagnostic, confirm the delivery of a therapy, record data, enter a communications session with an external device, or perform any function the IMD is capable of performing.

The switch is disposed on the casing of the housing, on a connector block, or on an edge portion of the housing. The force required to actuate the switch is set such that inadvertent pressure (e.g., lying down, wearing tight clothing) will not actuate the switch, yet the pressure required will not be so high that repeated actuation causes bruising or soreness to the surrounding tissue.

In one embodiment, the present invention is an implantable medical device comprising a housing having an interior and an exterior. The device also includes a switch disposed on the exterior of the housing.

In another embodiment, the present invention is an implantable medical device comprising means for physically communicating with the implantable medical device after implantation. In another embodiment, the present invention is an implantable medical device comprising a housing and processing means disposed within the housing. The device also includes switch means actuable external to the housing and in communication with the processing means

In another embodiment, the present invention is an implantable medical device comprising a hermetically sealed housing, a processor disposed within the housing, and a lead coupleable to the housing for delivering therapy initiated by the processor. The device also includes a hermetically sealed switch disposed on an external portion of the hermetically sealed housing and in communication with the processor.

The present invention also includes a method comprising applying pressure to tissue adjacent to an implanted medical device, wherein the application of pressure actuates a switch disposed on an exterior portion of the implanted medical device. The method further includes triggering an action within the implanted medical device based upon the actuation of the switch.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an illustration of a PCD type system according to the present invention.

FIG. 2 is a block, functional diagram of a PCD type device adapted to carry out the features of the present invention.

FIG. 3 is a perspective view of the external programming unit of FIG. 1.

FIG. 4 is a planar view of an implantable medical device with a switch incorporated into the housing.

FIG. 5 is a planar view of an implantable medical device with a switch incorporated into the connector block.

FIG. 6 is a planar view of an implantable monitoring device with a switch incorporated into the housing.

FIG. 7 is a schematic illustration of the IMD of FIG. 4 implanted within a patient.

### DETAILED DESCRIPTION

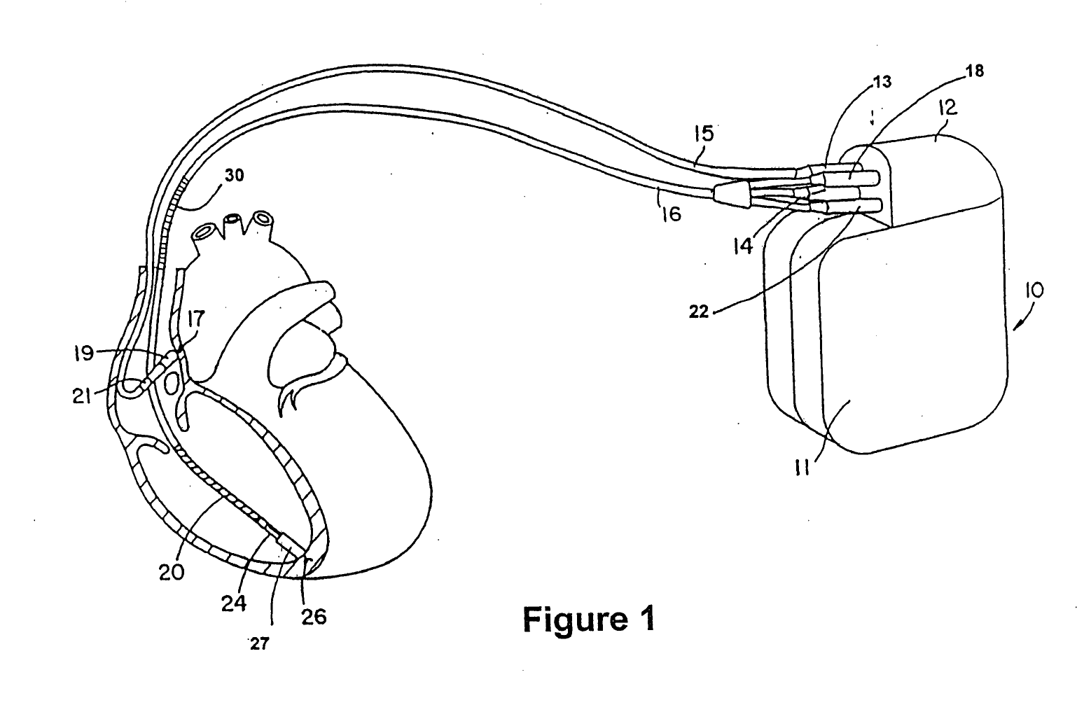

Referring now to FIG. 1, there are illustrated an IMD 10, exemplary illustrated as a defibrillator, and leads 15 and 16, making up a PCD (pacemaker cardioverter defibrillator) type system, representative of various implantable medical devices. The leads shown are illustrative, it being noted that other specific forms of leads are within the scope of this invention and that more or fewer leads may be employed depending upon the application. Ventricular lead 16 as illustrated has, located adjacent to the distal end, an extendable helix electrode 26 and a ring electrode 24, the helix electrode being mounted retractably within an insulative head 27. Electrodes 24 and 26 are used for bipolar ventricular pacing and for sensing ventricular depolarizations. While electrodes 24 and 26 may be used for bipolar pacing and sensing, electrode 26 may be used in conjunction with the surface of device casing 11, which surface acts as a common or indifferent electrode in what is termed unipolar operation. Ventricular lead 16 also carries a coil electrode 20, sometimes referred to as the RV (right ventricular) coil, for delivering defibrillation and/or cardioversion pulses. Coil electrode 20 is positioned on lead 16 so that when the distal tip is at the apex of the ventricle, coil 20 is positioned in the right ventricle. Lead 16 may also carry, optionally, an SCV coil 30, positioned in the subclavian vein, which can be used, for example, for R wave sensing and/or applying cardioversion pulses. Lead 16 carries respective concentric coil conductors (not shown), separated from one another by appropriate means such as tubular insulative sheaths and running the length of the lead for making electrical connection between the PCD device 10 and respective ones of electrodes 20, 24, 26 and 30.

Atrial lead 15 as illustrated includes an extendable helix electrode 17 and a ring electrode, the helix electrode being mounted retractably within an insulative head 19. Electrodes 17 and 21 are used for bipolar atrial pacing and for sensing atrial depolarizations. While electrodes 17 and 21 may be used for bipolar pacing and sensing, electrode 17 may be used in conjunction with the surface of device casing 10, which surface acts as a common or indifferent electrode in what is termed unipolar operation. Note that, in this example, atrial lead 15 is not equipped with coils for use in the sensing and delivery of cardioversion of defibrillation pulses. This is not meant to preclude the inclusion of such applications that may be used advantageously with the present invention.

PCD device 10, is shown in combination with atrial and ventricular leads, with the lead connector assembly 13, 14, 18, and 22 being inserted into the connector block 12 of the device 10. As used herein, the term “PCD type” device refers to any device that can apply both pacing therapy and shock therapy for controlling arrhythmias. It should be appreciated that the present invention is applicable to various IMDs including, but not limited to pacemakers, cardioverters, defibrillators, monitors, drug pumps, neural stimulators, muscular stimulators, spinal stimulators, or any combination thereof. Furthermore, the present invention may be practiced with IMDs such as device 10 that include attachable leads or with various devices, such as an implantable subcutaneous monitor that have electrodes within the housing and do not utilize external leads.

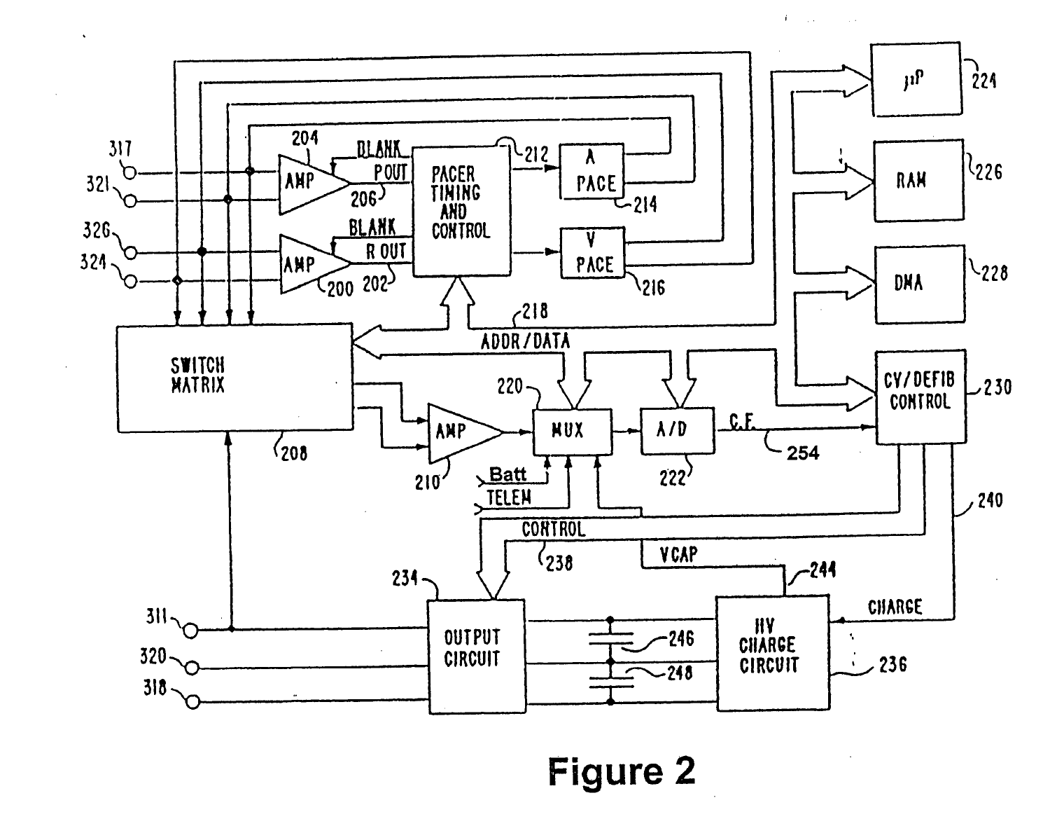

FIG. 2 is a functional schematic diagram of an implantable PCD in which the present invention may usefully be practiced. This diagram should be taken as exemplary of the type of device in which the invention may be embodied, and not as limiting, as it is believed that the invention may usefully be practiced in a wide variety of device implementations.

The device is provided with a lead system including electrodes, which may be as illustrated in FIG. 1. Alternate lead systems may of course be substituted. If the electrode configuration of FIG. 1 is employed, the correspondence to the illustrated electrodes is as follows. Electrode 311 corresponds to electrode 16, and is the uninsulated portion of the housing of the implantable pacemaker/cardioverter/defibrillator. Electrode 320 corresponds to electrode 20 and is a defibrillation electrode located in the right ventricle. Electrode 318 corresponds to electrode 30 and is a defibrillation electrode located in the superior vena cava. Electrodes 324 and 326 correspond to electrodes 24 and 26, and are used for sensing and pacing in the ventricle. Electrodes 317 and 321 correspond to electrodes 17 and 21 and are used for pacing and sensing in the atrium.

Electrodes 311, 318 and 320 are coupled to high voltage output circuit 234. Electrodes 324 and 326 are located on or in the ventricle and are coupled to the R-wave amplifier 200, which preferably takes the form of an automatic gain controlled amplifier providing an adjustable sensing threshold as a function of the measured R-wave amplitude. A signal is generated on R-out line 202 whenever the signal sensed between electrodes 324 and 326 exceeds the present sensing threshold.

Electrodes 317 and 321 are located on or in the atrium and are coupled to the P-wave amplifier 204, which preferably also takes the form of an automatic gain controlled amplifier providing an adjustable sensing threshold as a function of the measured P-wave amplitude. A signal is generated on P-out line 206 whenever the signal sensed between electrodes 317 and 321 exceeds the present sensing threshold.

Switch matrix 208 is used to select which of the available electrodes are coupled to amplifier 210 for use in digital signal analysis. Selection of electrodes is controlled by the microprocessor 224 via data/address bus 218, which selections may be varied as desired. Signals from the electrodes selected for coupling to bandpass amplifier 210 are provided to multiplexer 220, and thereafter converted to multi-bit digital signals by A/D converter 222, for storage in random access memory 226 under control of direct memory access circuit 228. Microprocessor 224 may employ digital signal analysis techniques to characterize the digitized signals stored in random access memory 226 to recognize and classify the patient's heart rhythm employing any of the numerous signal-processing methodologies known to the art.

The remainder of the circuitry is dedicated to the provision of cardiac pacing, cardioversion and defibrillation therapies, and, for purposes of the present invention may correspond to known circuitry. An exemplary apparatus is disclosed of accomplishing pacing, cardioversion and defibrillation functions follows. The pacer timing/control circuitry 212 includes programmable digital counters which control the basic time intervals associated with DDD, VVI, DVI, VDD, AAI, DDI and other modes of single and dual chamber pacing well known to the art. Circuitry 212 also controls escape intervals associated with anti-tachyarrhythmia pacing in both the atrium and the ventricle, employing any anti-tachyarrhythmia pacing therapies known to the art.

Intervals defined by pacing circuitry 212 include atrial and ventricular pacing escape intervals, the refractory periods during which sensed P-waves and R-waves will not restart the escape pacing interval timing. The durations of these intervals are determined by microprocessor 224, in response to stored data in memory 226 and are communicated to the pacing circuitry 212 via address/data bus 218. Pacer circuitry 212 also determines the amplitudes and pulse widths of the cardiac pacing pulses under control of microprocessor 224.

During pacing, the escape interval timers within pacer timing/control circuitry 212 are reset upon sensing of R-waves and P-waves as indicated by signals on lines 202 and 206, and in accordance with the selected mode of pacing on timeout trigger generation of pacing pulses by pacer output circuitry 214 and 216, which are coupled to electrodes 317, 321, 324 and 326. The escape interval timers are also reset on generation of pacing pulses, and thereby control the basic timing of cardiac pacing functions, including anti-tachyarrhythmia pacing. The durations of the intervals defined by the escape interval timers are determined by microprocessor 224, via data/address bus 218. The value of the count present in the escape interval timers when reset by sensed R-waves and P-waves may be used to measure the durations of R-R intervals, P-P intervals, P-R intervals, and R-P intervals, which measurements are stored in memory 226 and used in conjunction with the present invention to diagnose the occurrence of a variety of tachyarrhythmias, as discussed in more detail below.

Microprocessor 224 operates as an interrupt driven device, and is responsive to interrupts from pacer timing/control circuitry 212 corresponding to the occurrences of sensed P-waves and R-waves and corresponding to the generation of cardiac pacing pulses. These interrupts are provided via data/address bus 218. Any necessary mathematical calculations to be performed by microprocessor 224 and any updating of the values or intervals controlled by pacer timing/control circuitry 212 take place following such interrupts. A portion of the memory 226 may be configured as a plurality of recirculating buffers, capable of holding series of measured intervals, which may be analyzed in response to the occurrence of a pace or sense interrupt to determine whether the patient's heart is presently exhibiting atrial or ventricular tachyarrhythmia.

The arrhythmia detection method of the PCD may include prior art tachyarrhythmia detection algorithms. As described below, the entire ventricular arrhythmia detection methodology of presently available Medtronic pacemaker/cardioverter/defibrillators is employed as part of the arrhythmia detection and classification method according to the disclosed preferred embodiment of the invention. However, any of the various arrhythmia detection methodologies known to the art, as discussed in the Background of the Invention section above might also be usefully employed in alternative embodiments of the implantable PCD.

In the event that an atrial or ventricular tachyarrhythmia is detected, and an anti-tachyarrhythmia pacing regimen is desired, appropriate timing intervals for controlling generation of anti-tachyarrhythmia pacing therapies are loaded from microprocessor 224 into the pacer timing and control circuitry 212, to control the operation of the escape interval timers therein and to define refractory periods during which detection of R-waves and P-waves is ineffective to restart the escape interval timers.

In the event that generation of a cardioversion or defibrillation pulse is required, microprocessor 224 employs the escape interval timer to control timing of such cardioversion and defibrillation pulses, as well as associated refractory periods. In response to the detection of atrial or ventricular fibrillation or tachyarrhythmia requiring a cardioversion pulse, microprocessor 224 activates control circuitry 230, which initiates charging of the high voltage capacitors 246, 248 via charging circuit 236, under control of high voltage charging control line 240 242. The voltage on the high voltage capacitors is monitored via VCAP line 244, which is passed through multiplexer 220 and in response to reaching a predetermined value set by microprocessor 224, results in generation of a logic signal on Cap Full (CF) line 254, terminating charging. Thereafter, timing of the delivery of the defibrillation or cardioversion pulse is controlled by pacer timing/control circuitry 212. Following delivery of the fibrillation or tachycardia therapy the microprocessor then returns the device to cardiac pacing and awaits the next successive interrupt due to pacing or the occurrence of a sensed atrial or ventricular depolarization.

In the illustrated device, delivery of the cardioversion or defibrillation pulses is accomplished by output circuit 234, under control of control circuitry 230 via control bus 238. Output circuit 234 determines whether a monophasic or biphasic pulse is delivered, whether the housing 311 serves as cathode or anode and which electrodes are involved in delivery of the pulse.

In modern implantable cardioverter/defibrillators, the physician, from a menu of therapies that are typically provided, programs the specific therapies into the device. For example, on initial detection of an atrial or ventricular tachycardia, an anti-tachycardia pacing therapy may be selected and delivered to the chamber in which the tachycardia is diagnosed or to both chambers. On redetection of tachycardia, a more aggressive anti-tachycardia pacing therapy may be scheduled. If repeated attempts at anti-tachycardia pacing therapies fail, a higher energy cardioversion pulse may be selected for subsequent delivery. Therapies for tachycardia termination may also vary with the rate of the detected tachycardia, with the therapies increasing in aggressiveness as the rate of the detected tachycardia increases. For example, fewer attempts at anti-tachycardia pacing may be undertaken prior to delivery of cardioversion pulses if the rate of the detected tachycardia is below a preset threshold.

In the event that fibrillation is identified, the typical therapy will be the delivery of a high amplitude defibrillation pulse, typically in excess of 5 joules. Lower energy levels may be employed for cardioversion. As in the case of currently available implantable pacemakers/cardioverter/defibrillators, it is envisioned that the amplitude of the defibrillation pulse may be incremented in response to failure of an initial pulse or pulses to terminate fibrillation.

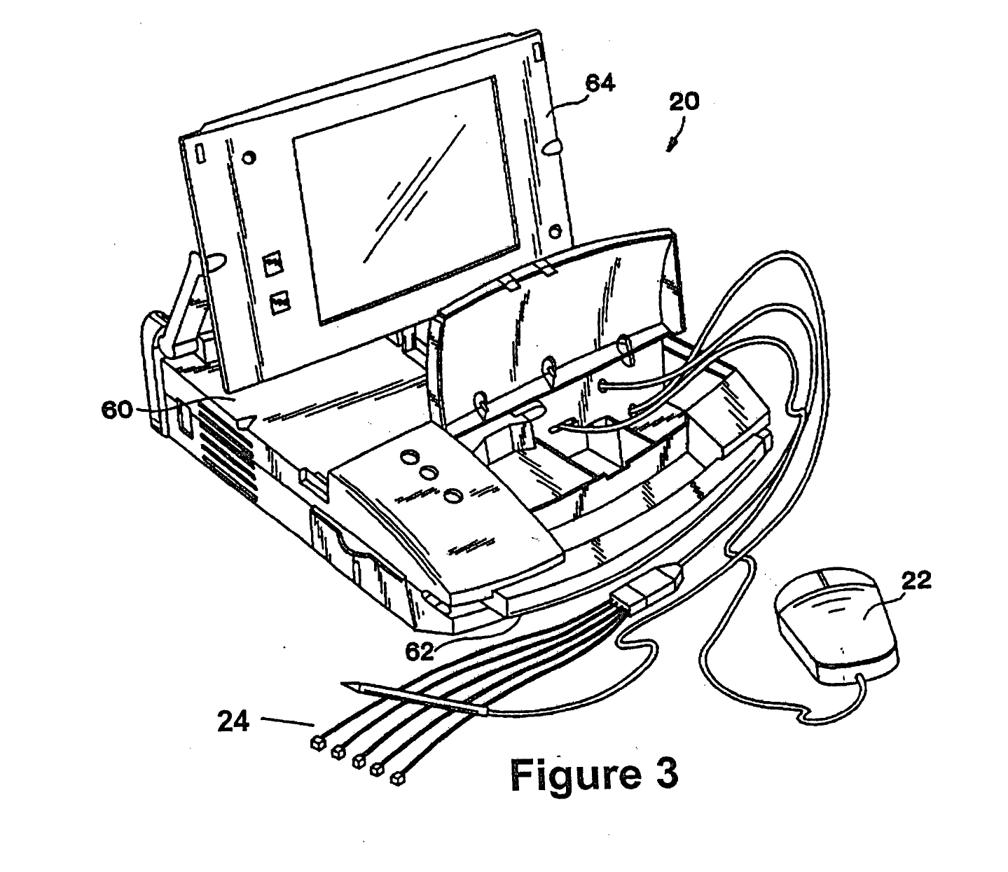

FIG. 3 is a perspective view of programming unit program 20. Internally, programmer 20 includes a processing unit (not shown in the Figure) that is a personal computer type motherboard, e.g., a computer motherboard including an Intel Pentium 3 microprocessor and related circuitry such as digital memory. The details of design and operation of the programmer's computer system will not be set forth in detail in the present disclosure, as it is believed that such details are well-known to those of ordinary skill in the art.

Referring to FIG. 3, programmer 20 comprises an outer housing 60, which is preferably made of thermal plastic or another suitably rugged yet relatively lightweight material. A carrying handle, designated generally as 62 in FIG. 2, is integrally formed into the front of housing 60. With handle 62, programmer 20 can be carried like a briefcase.

An articulating display screen 64 is disposed on the upper surface of housing 60. Display screen 64 folds down into a closed position (not shown) when programmer 20 is not in use, thereby reducing the size of programmer 20 and protecting the display surface of display 64 during transportation and storage thereof.

A floppy disk drive is disposed within housing 60 and is accessible via a disk insertion slot (not shown). A hard disk drive is also disposed within housing 60, and it is contemplated that a hard disk drive activity indicator, (e.g., an LED, not shown) could be provided to give a visible indication of hard disk activation.

As would be appreciated by those of ordinary skill in the art, it is often desirable to provide a means for determining the status of the patient's conduction system. Normally, programmer 20 is equipped with external ECG leads 24.

In accordance with the present invention, programmer 20 is equipped with an internal printer (not shown) so that a hard copy of a patient's ECG or of graphics displayed on the programmer's display screen 64 can be generated. Several types of printers, such as the AR-100 printer available from General Scanning Co., are known and commercially available.

In the perspective view of FIG. 3, programmer 20 is shown with articulating display screen 64 having been lifted up into one of a plurality of possible open positions such that the display area thereof is visible to a user situated in front of programmer 20. Articulating display screen is preferably of the LCD or electro-luminescent type, characterized by being relatively thin as compared, for example, a cathode ray tube (CRT) or the like.

As would be appreciated by those of ordinary skill in the art, display screen 64 is operatively coupled to the computer circuitry disposed within housing 60 and is adapted to provide a visual display of graphics and/or data under control of the internal computer.

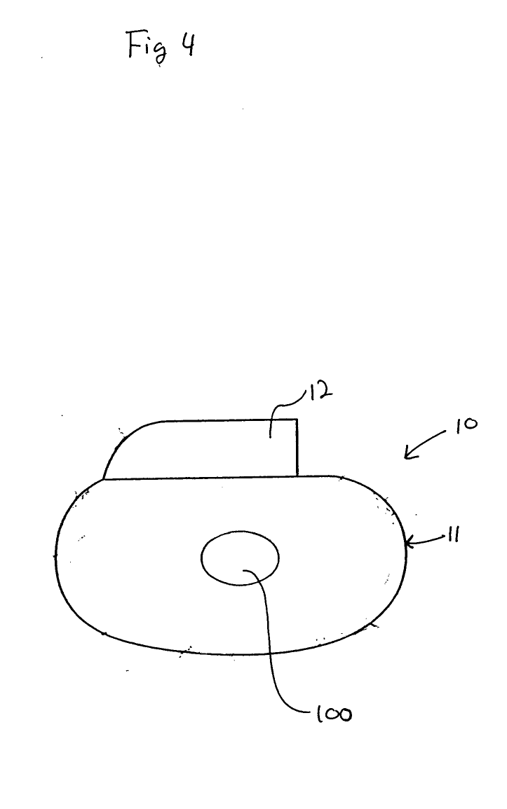

FIG. 4 is a planar view of IMD 10. As previously described, IMD 10 includes a housing having the hermetically sealed casing 11 and connector block 12. A hermitically sealed switch 100 is located within the casing 11. In one embodiment, switch 100 is a momentary switch that makes contact (or breaks contact) when pushed. Other types of switches such as a toggle on/off type switch could be used. Once implanted, the switch 100 is actuated by applying pressure to the tissue over the implant site. With sufficient pressure the switch 100 is actuated and a predetermined action is initiated. The switch 100 may have an identifying physical feature such as a raised profile, bump, or depression or cavity, to facilitate location of the switch by the patient with palpitation prior to forcefully activating the switch by pressing on it.

In this manner, the patient can initiate certain actions within IMD 10 without requiring the use of an external device, such as a programmer, magnet, RF transceiver or the like. Thus, the action can be taken at any time and provides an additional level of freedom of operation to the patient.

The actions taken by actuating switch 100 include most capabilities of the IMD 10. The action of the switch 100 may depend on the duration of time that the switch is depressed. By way of example, such actions include inhibiting the delivery of a therapy. As previously described, the IMD 10 may determine that a particular therapy is appropriate, e.g., defibrillation for atrial fibrillation; however, the patient may prefer to wait an extended period of time to allow the rhythm to stabilize on its own. Thus, in this example, actuating the switch 100 causes the IMD 10 to inhibit the delivery of a therapy. The inhibited therapy could be any therapy that the patient can safely choose to forego based on personal comfort. Conversely, actuation of the switch 100 initiates a therapy or increases a level of therapy, again based on the patient's personal comfort level.

Actuation of the switch 100, in another embodiment, queries the IMD 10 for a status or to perform a self-diagnostic. Thus, the patient can actuate the switch 100 and then receive a confirmation that the IMD 10 is operable. Such a confirmation could be delivered by tactile stimulation (e.g., vibration), the generation of certain sounds, tones or alarms, by sending a signal to an external device (e.g., a programmer), or through any other communication platform. Likewise, the patient could query the device to determine whether a particular therapy had been delivered. For example, the patient may wish to determine if a perceived shock was really delivered or if it was a phantom shock.

In yet another embodiment, actuation of the switch 100 causes the IMD 10 to record data. Such data includes, for example, a date and time stamp indicative of when the patient felt symptoms. Alternatively, actuation of the switch 100 causes the IMD 10 to record physiological data from a predetermined time frame. That is, the IMD 10 continuously monitors such data, but only records that data when the patient indicates, through actuation of switch 100, that symptoms have been detected. This is advantageous in that the patient can cause the IMD 10 to record data at any time, without requiring the use of an external actuator that can be lost, forgotten, or inconveniently located. For retrieval of data, actuation of the switch 100 could be programmed, in one embodiment, to initiate a telemetry session with a remote device and facilitate data transfer.

Upon actuation, the switch, in one embodiment, provides an indication of actuation. For example, the switch 100 provides tactile feedback when fully depressed, such as a “clicking” sensation. Alternatively, a sound, vibration, or other perceivable alert could be generated to indicate that the switch 100 has been actuated.

While many actions actuable by switch 100 are implemented by a single deployment of the switch 100, the present invention is not so limited. That is, more complex commands can be delivered to the IMD 10 through a series of switch actuations. For example, depressing switch 100 a multiple number of times during a predetermined time period causes a different action that simply actuating the switch 100 once. As can be imagined, various combinations of timing and the number of actuations can be utilized to communicate a wide variety of information to the IMD 10. Also, the duration of switch (e.g., push and hold for some predetermined period, e.g., one to three seconds) contact may encode information and become a different command. By way of example, an initial deployment of the switch 100 inhibits the delivery of a therapy. Subsequent deployment of the switch 100 indicates a time interval. For example, the second actuation causes inhibition for five minutes, the third another five minutes (a total of ten minutes), and so on.

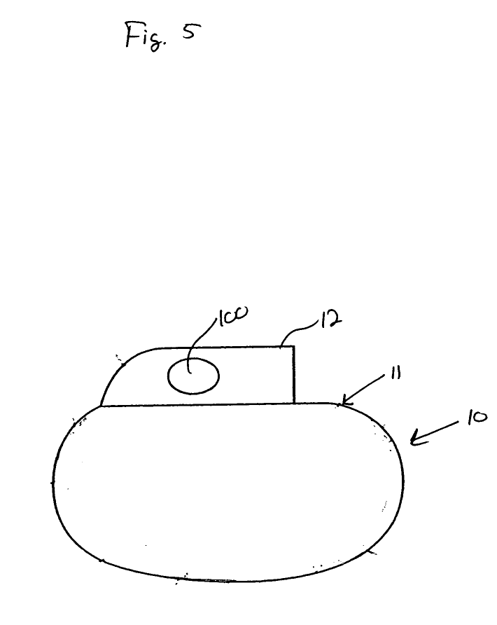

Switch 100 can take various forms so long as a hermetic seal is maintained. For example, switch 100 is a membrane switch disposed within the housing 11 or “can” of the IMD 10. FIG. 5 illustrates the switch 100 disposed within the connector block 12 of the IMD 10. When IMD 10 is implanted subcutaneously, the switch 100 is positioned on the housing 11. For submuscular or submammary implants, the switch 100 may be mounted on the connector block 12 or along the edge of the housing 11, to facilitate actuation.

The amount of force required to actuate the switch 100 should be chosen to facilitate patient actuation while minimizing accidental actuation. For example, the force required should be sufficiently high so that a patient lying on their chest or wearing tight clothing will not inadvertently cause the switch 100 to actuate. Conversely, the force required should not be so high that deployment of the switch 100 causes pain, discomfort, or bruising.

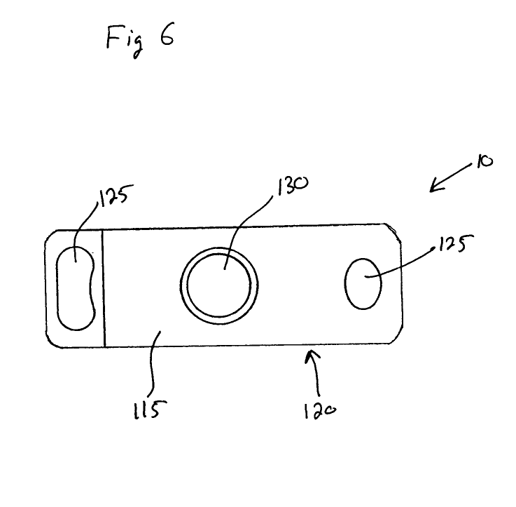

While the switch 100 has been described in the context of a pacemaker/defibrillator/cardioverter, the switch 100 can be utilized in a wide variety implantable medical devices such as, muscle stimulators, neural stimulators, drug pumps and the like. FIG. 6 is a planar view of an IMD 10 in the form of an implantable cardiac monitor 120, with an externally actuable switch 130 incorporated thereon. Monitor 120 includes a hermetically sealed housing 115 having multiple electrodes 125 for sensing cardiac signals. Once implanted, external actuation of the switch 139 causes predetermined results. For example, actuation of the switch 130 could toggle the device on and off. Alternatively, actuation could query the device as to its status and a signal could be generated if the monitor 120 is functioning properly. In another example, actuation of the switch 130 could cause certain information to be recorded such as the date and time or the cardiac data sensed for a predetermined time period could be stored in memory. As another example, actuation of the switch 130 could cause the monitor 120 to begin a telemetry session and to uplink to an external device.

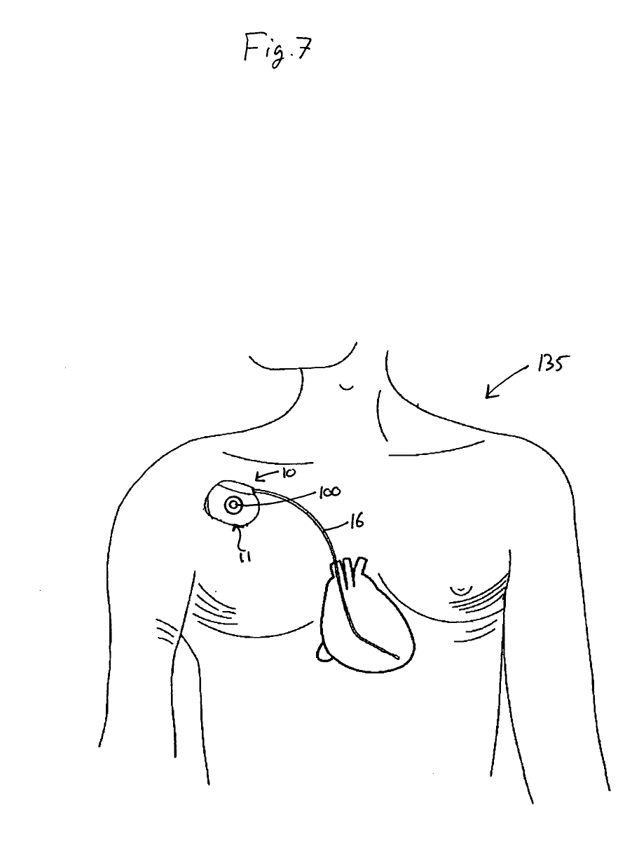

FIG. 7 is a schematic illustration of IMD 10 implanted within a patient 135. The patient 135 is aware of the relative position of the IMD 10 beneath the skin and/or muscle. Thus, when appropriate, the patient 135 presses one or more fingers against the tissue, which in turn contacts the housing 11 of the IMD 10. With sufficient force, this action will actuate the switch 100. Preferably, the patient 135 is alerted when the switch 100 is successfully actuated. For example, the switch 100 may provided a clicking sensation when deployed. Alternatively, a sound or other perceivable alert may be generated by the IMD simply as an alert that the switch 100 has been actuated.

Depending upon the programmed action of the switch 100, various safety protocols may be implemented. For example, if inadvertent actuation of the switch 100 could cause a serious consequence, IMD can be programmed so that a single actuation is insufficient to trigger the action. With the generation of a sound or other perceivable alert, the patient 135 is notified that the switch 100 is being inadvertently actuated and corrective action can be taken. With such a protocol, the patient 135 may be required to actuate switch 100 in a predetermined sequence or a specific number of times within a predetermined time frame to initiate the desired action.

Although the present invention has been described with reference to preferred embodiments, persons skilled in the art will recognize that changes may be made in form and detail without departing from the spirit and scope of the invention.

1. An implantable cardiac medical device comprising:
a housing having an interior and an exterior;
a sensor for sensing cardiac signals
a processor disposed within the housing and in communication with the sensor for processing the cardiac signals from the sensor; and
a switch disposed on the exterior of the housing.

2. The implantable medical device of claim 1 further comprising a component within the interior of the housing, wherein the switch is in communication with the component.

3. The implantable medical device of claim 1, wherein the switch is hermetically sealed with respect to the housing.

4. The implantable medical device of claim 1, wherein the housing further comprises a connector block and the switch is disposed on the connector block.

5. The implantable medical device of claim 1, wherein actuation of the switch causes the implantable medical device to inhibit a therapy.

6. The implantable medical device of claim 1, wherein actuation of the switch causes the implantable medical device to initiate a therapy.

7. The implantable medical device of claim 1, wherein actuation of the switch causes the implantable medical device to adjust a therapy.

8. The implantable medical device of claim 1, wherein actuation of the switch causes the implantable medical device to record data.

9. The implantable medical device of claim 8, wherein the data includes a time and a date.

10. The implantable medical device of claim 8, wherein the data includes sensor data.

11. The implantable medical device of claim 1, wherein actuation of the switch causes the implantable medical device to perform a self-diagnostic.

12. The implantable medical device of claim 1, wherein actuation of the switch causes the implantable medical device to enter a communications session with an external device.

13. The implantable medical device of claim 1, wherein the implantable medical device is a pacemaker.

14. The implantable medical device of claim 1, wherein the implantable medical device is a defibrillator.

15. The implantable medical device of claim 1, wherein the implantable medical device is an implantable cardiac monitor.

16. An implantable medical device comprising means for physically communicating with the implantable medical device after implantation.

17. An implantable medical device comprising;
a housing;
processing means disposed within the housing; and
switch means actuable external to the housing and in communication with the processing means.

18. An implantable cardiac medical device comprising:
a hermetically sealed housing;
a processor disposed within the housing;
a lead coupleable to the housing for delivering cardiac therapy initiated by the processor; and
a hermetically sealed switch disposed on an external portion of the hermetically sealed housing and in communication with the processor.

19. A method comprising:
applying pressure to tissue adjacent to an implanted cardiac medical device, wherein the application of pressure actuates a switch disposed on an exterior portion of the implanted cardiac medical device; and
triggering an action within the implanted cardiac medical device based upon the actuation of the switch.

20. The method of claim 19, wherein the action is one of the following: inhibiting a cardiac therapy, initiating a cardiac therapy, recording cardiac data, performing a self diagnostic of the cardiac medical device, or entering a communication session with an external device.
